(19) United States
(12) Patent Application Publication
(10) Pub. No.: US 2006/0040401 A1
(43) Pub. Date: Feb. 23, 2006

(54) Method and equipment for measuring the concentration of antiseptic solution

(75) Inventors: Jun Watabe, Shizuoka (JP); Tetsuo Amano, Shizuoka (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Amano Corporation, Iwata-shi (JP)

(21) Appl. No.: 11/258,948

(22) Filed: Oct. 27, 2005

(30) Foreign Application Priority Data
Aug. 27, 2003 (JP) 2003-302830

(51) Int. Cl. G06F 19/00; G01N 33/00

(52) U.S. Cl. 436/128; 702/22

(57) ABSTRACT

[Problem] To provide an antiseptic-solution concentration measuring method and equipment for easy, simple, economical and accurate measurement of the concentration of a glutaral, phtharal or similar antiseptic solution.

[Means for Solving Problem] The temperature of the antiseptic solution S, such as the glutaral or phtharal solution, is measured by a thermometer, and at the same time the ultraviolet absorbance of the antiseptic solution is electrically detected by a light-receiving part 9, then a function of proportionality between the measured ultraviolet absorbance of the antiseptic solution and pre-measured ultraviolet absorbance of an undiluted antiseptic solution and a function of proportionality between the ultraviolet absorbance and concentration of said undiluted antiseptic solution are calculated by a computer to measure the concentration of the antiseptic solution S.

### TECHNICAL FIELD

The present invention relates generally to a technique for deciding whether the concentration of a repeatedly used antiseptic solution, such as a glutaral or phtharal solution, is adequate for reuse and, more particularly, to method and equipment for quantitatively detecting the concentration of such antiseptic solutions.

### BACKGROUND ART

At present, the glutaral and phtharal solutions, which are high-level antiseptic solutions widely used in hospitals or similar medical facilities, are not disposed of after each use but instead they are usually stored for reuse in many cases. Accordingly, these antiseptic solutions are inevitably deteriorated by secular changes after opening their sealed packages or they are diluted by the mixing thereinto of water that often adheres to objects of disinfection during their cleaning and rinsing prior to disinfection—this lowers the concentrations of components effective for disinfection, often resulting in a failure of appropriate disinfection. Hence, the use of such antiseptic solutions calls for accurate measurement of their concentrations.

Conventionally, the concentration of such antiseptic solution is measured by colorimetry using test strips offered by antiseptic-solution makers, spectrometry, liquid chromatography, or the like. The colorimetry using test strips is simple and hence easy, but it is very difficult with this method to measure the concentration of the antiseptic solution on the borderline between adequate or low; in some cases, the use of the antiseptic solution is abandoned for safety reasons despite its adequate concentration, or an error in measurement may sometimes allow the use of an antiseptic solution of a concentration lower than the adequate value. The spectrometry and the liquid chromatography are both defective in that their measuring equipment is expensive and difficult to handle and consumes much time for measurement. From the economical point of view, it is disadvantageous to install such measuring equipment in hospitals or similar medical facilities for only measuring the concentration of the antiseptic solution.

### DISCLOSURE OF THE INVENTION

### Problem to be Solved by the Invention

The present invention is intended to solve the above-described problems of the prior art, and has for its object to provide a method and equipment for easy, simple, economical and accurate measurement of concentrations of glutaral, phtharal and similar antiseptic solutions.

### Means for Solving the Problem

To attain the above objective, the method for measuring the concentration of an antiseptic solution according to claim 1 of this application is characterized by the steps of electrically measuring the absorbance of the antiseptic solution, such as a glutaral or phtharal solution, by a photodetector to detect its absorbance of ultraviolet rays; and calculating, by a computer, a function of proportionality between the measured ultraviolet absorbance of the antiseptic solution and pre-measured ultraviolet absorbance of an undiluted antiseptic solution and a function of proportionality between the ultraviolet absorbance and concentration of the undiluted antiseptic solution.

The method for measuring the concentration of an antiseptic solution according to claim 2 of this application is characterized by: measuring the temperature of the antiseptic solution, such as a glutaral or phtharal solution, by a thermometer; electrically measuring the absorbance of the antiseptic solution by a photodetector to detect its absorbance of ultraviolet rays; and calculating, by a computer, a function of proportionality between the ultraviolet absorbance of the antiseptic solution and pre-measured ultraviolet absorbance of an undiluted antiseptic solution and a function of proportionality between the ultraviolet absorbance and concentration of the undiluted antiseptic solution.

The method for measuring the concentration of an antiseptic solution according to claim 3 of this application, which is a modification of the method of claim 1 or 2, is characterized by the steps of: pouring an unused antiseptic solution and pure water into the optical cell separately of each other; calibrating, by a computer, electrical signals from the optical cell which represent the ultraviolet absorbance and concentration of the undiluted antiseptic solution and the pure water, respectively; and calibrating concentration measuring equipment based on the calibrated values.

Equipment for measuring the concentration of an antiseptic solution according to claim 4 of this application is characterized by: an optical cell for containing an antiseptic solution under measurement; a light-emitting part for irradiating the antiseptic solution in said optical cell with ultraviolet rays; a light-receiving part provided outside the optical cell, for receiving ultraviolet rays partly absorbed by the antiseptic solution in proportion to the concentration thereof, for detecting the ultraviolet absorbance of the antiseptic solution from the intensity of the received ultraviolet rays, and for generating an electrical signal representing the ultraviolet absorbance of the antiseptic solution; a computer for calculating the concentration value of the antiseptic solution by calculating the relationships between the electrical signal and pre-input electrical signals representing the ultraviolet absorbance and concentration of an undiluted antiseptic solution; and a display for displaying the concentration value calculated by the computer.

Equipment for measuring the concentration of an antiseptic solution according to claim 5 of this application is characterized by: an optical cell for containing an antiseptic solution under measurement; a light-emitting part for irradiating the antiseptic solution in the optical cell with ultraviolet rays; a light-receiving part provided outside the optical cell, for receiving ultraviolet rays partly absorbed by said antiseptic solution in proportion to the concentration thereof, for detecting the ultraviolet absorbance of the antiseptic solution from the intensity of the received ultraviolet rays, and for generating an electrical signal representing the ultraviolet absorbance of the antiseptic solution; a thermometer for measuring the temperature of the antiseptic solution in the optical cell, and for converting said measured temperature to the corresponding electrical signal; a computer for calculating the concentration value of said antiseptic solution by calculating the relationships between the electrical signals representing the ultraviolet absorbance and temperature of the antiseptic solution and pre-input electrical signals representing the absorbance and concentration of an undiluted antiseptic solution; and a display for displaying the concentration value calculated by the computer.

The equipment for measuring the concentration of an antiseptic solution according to claim 6 of this application, which is a modification of claim 4 or 5, is characterized in that: an unused antiseptic solution and pure water are separately poured into the optical cell; electrical signals from the optical cell which represent the ultraviolet absorbance and concentration of the unused antiseptic solution and the pure water, respectively, are calibrated by the computer; and the measuring equipment is calibrated accordingly.

The measurement of the concentration of the antiseptic solution according to the method of the present invention begins with electrical detection of the ultraviolet absorbance of the antiseptic solution by a photodetector. Then, the concentration of the antiseptic solution is accurately detected from the function of proportionality between the measured ultraviolet absorbance of the antiseptic solution and pre-measured ultraviolet absorbance of an undiluted antiseptic solution and the function of proportionality between the ultraviolet absorbance and concentration of the undiluted antiseptic solution.

Further, measurement of the temperature of the antiseptic solution by a thermometer is followed by electrical detection of the ultraviolet absorbance of the antiseptic solution. And, the function of proportionality between the measured ultraviolet absorbance of the antiseptic solution and pre-measured ultraviolet absorbance of an undiluted antiseptic solution and the proportional relation between the ultraviolet absorbance and concentration of the undiluted antiseptic solution are calculated by a computer to accurately detect the concentration of the antiseptic solution.

Prior to the measurement of concentration, an unused antiseptic solution and pure water are poured into the optical cell, and electrical signals from the optical cell, corresponding to the unused antiseptic solution and the pure water, respectively, are used to calibrate concentration measuring equipment. This ensures accurate measurement of the concentration of he antiseptic solution without being affected by external disturbance.

According to the antiseptic-solution concentration measuring equipment of the present invention, the antiseptic solution under measurement is poured into an optical cell, and the ultraviolet absorbance of the antiseptic solution is detected by a photodetector. That is, the photodetector receives ultraviolet rays partly absorbed by the antiseptic solution in proportion to its concentration, then detects the ultraviolet absorbance of the antiseptic solution, and outputs an electrical signal representative of the concentration of the antiseptic solution. The temperature of the antiseptic solution in the electrical cell is measured, as required, by a thermometer and converted to the electrical signal corresponding to the measured temperature. The concentration of the antiseptic solution is calculated by the computer from the relationship between the pre-input electrical signals indicating the ultraviolet absorbance and concentration of the undiluted antiseptic solution, coupled with the electrical signal fed from the thermometer, and the concentration thus detected is displayed on the display.

Prior to the measurement of concentration, an unused antiseptic solution and pure water are poured into the optical cell, and the electrical signals fed therefrom corresponding to antiseptic solution and the pure water are used to calibrate the measuring equipment to ensure accurate measurement of the concentration of the antiseptic solution without being affected by external fluctuation or disturbance.

### Effect of the Invention

The present invention permits easy and simple measurement of the concentration of an antiseptic solution and miniaturization of the equipment therefor as compared with the conventional spectrometry. Furthermore, the present invention permits reduction of the amount of antiseptic solution under measurement, and hence suppresses exposure by the antiseptic solution. With the thermometer mounted on the concentration measuring equipment, it is possible to correct and calibrate the temperature characteristic or dependence of the ultraviolet absorbance of the antiseptic solution. Moreover, the calibration of the measuring equipment by use of an unused antiseptic solution ensures high and reliable measurement accuracy.

### BEST MODE FOR CARRYING OUT THE INVENTION

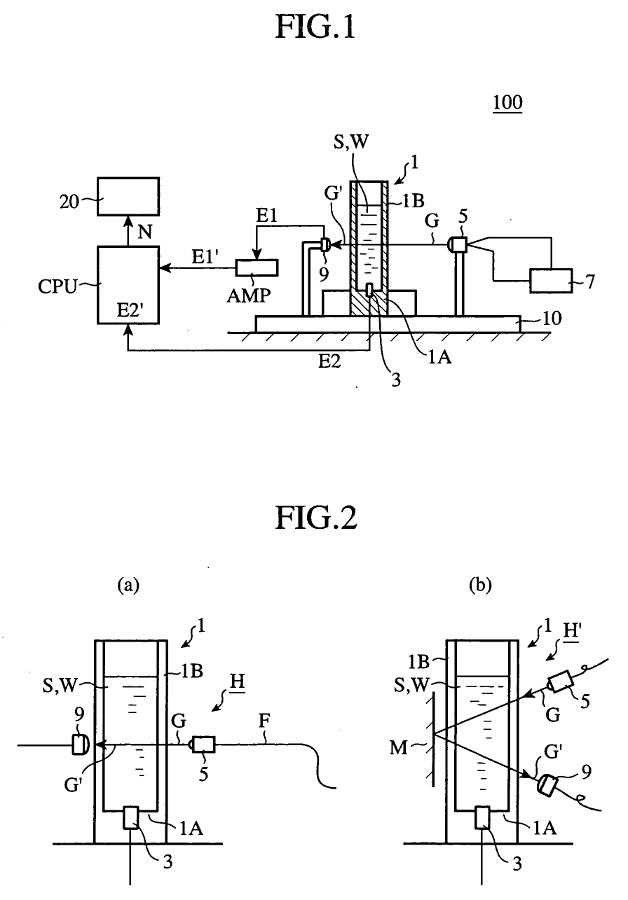

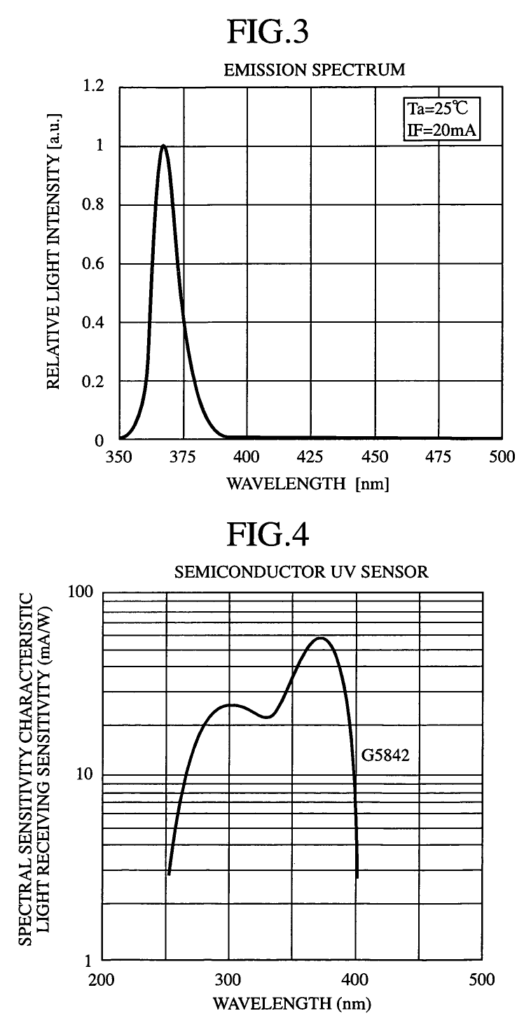

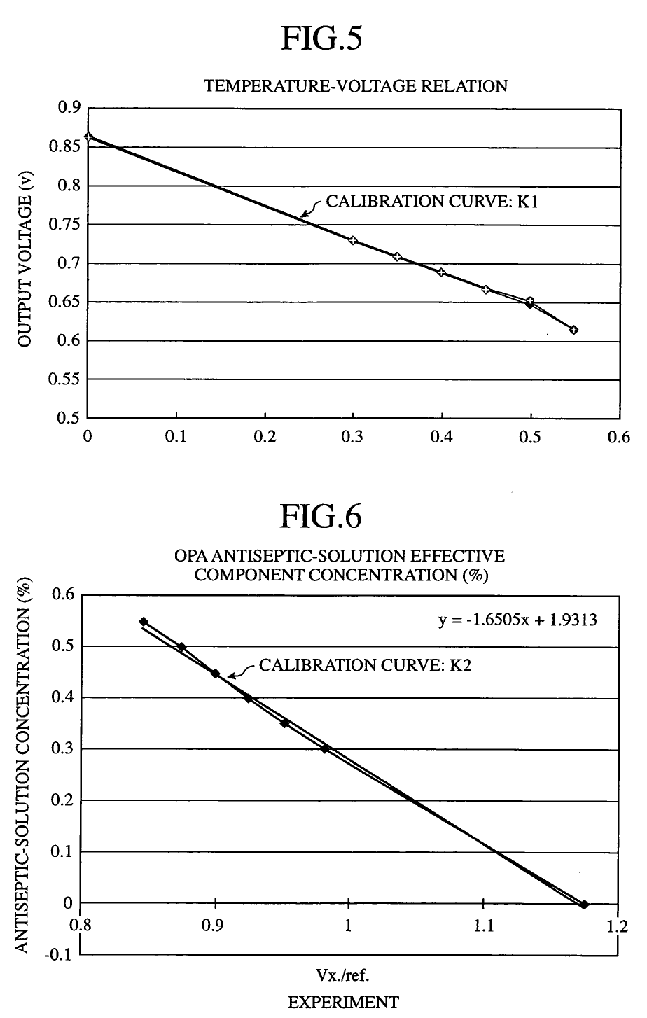

A description will be given, with respect to the accompanying drawings, of the method and apparatus for measuring the concentration of an antiseptic solution according to Embodiment 1 of the present invention. FIG. 1 illustrates in block form an apparatus 100 for measuring the concentration of an antiseptic solution according to the present invention, and FIG. 2 shows in section different arrangements of light-emitting and light-receiving parts set in an optical cell. FIG. 3 is graph showing the spectrum of light emitted from the light-emitting part, and FIG. 4 is a graph showing the spectral sensitivity of the light-receiving part. FIG. 5 is a graph showing the relationship between the concentration of an antiseptic solution and the output voltage from the light-receiving part, and FIG. 6 is a graph showing the relationship between recorded output values and the concentration of the antiseptic solution.

Referring first to FIG. 1, a description will be given of the general configuration of the antiseptic solution concentration measuring apparatus 100 according to Embodiment 1. On a measuring table 10 there is mounted an optical cell 1, which is filled with an antiseptic solution S whose concentration is to be measured; the temperature of the antiseptic solution under measurement is measured by a thermometer 3 placed on the bottom 1A of the optical cell, and the measured temperature is provided to a computer CPU described later on. A light-emitting part 5 is provided to irradiate the antiseptic solution S in the optical cell 1 with ultraviolet rays, and a power supply part 7 is connected to the light-emitting part 5 to excite it. On the measuring table 10 there is further mounted a light-receiving part 9, which receives ultraviolet rays G′ through the antiseptic solution S that absorbs a portion of the ultraviolet rays G from the light-emitting part 5 in proportion to the concentration of the antiseptic solution S. The light-receiving part 9 generates an electric signal E1 depending on the intensity of the ultraviolet rays being received and representative of the ultraviolet absorbance of the antiseptic solution S under measurement. The electric signal E1 is amplified by an amplifier AMP and then applied to the computer CPU. Based on the relationships between the electrical signal E1 from the amplifier AMP and pre-input signals indicating the concentration of an undiluted antiseptic solution and pure water, the computer CPU calculates the concentration of the antiseptic solution S under measurement and applies the calculated value N to a display 20. In this instance, an unused antiseptic solution S and pure water W are poured into the optical cell 1, respectively, and electrical signals E1′ representing their ultraviolet absorbance values and electrical signals E2′ indicating their temperatures are provided to the computer CPU, which uses them to calibrate the measuring equipment.

A detailed configuration and function of the concentration measuring apparatus 100 will be described below in more detail. As shown in FIGS. 1 and 2(a), the optical call 1 is formed by a transparent container 1B, which has a capacity large enough to contain the antiseptic solution S in an amount of approximately 5 to 10 CC. On both sides of the optical cell 1 there are disposed the light-emitting part 5 and the light-receiving part 9 which constitute detecting means H. The light-receiving part 9 receives the ultraviolet rays G′ through the antiseptic solution S that absorbs a portion of the ultraviolet rays G from the light-emitting part 5 in proportion to the concentration of the antiseptic solution S. In FIG. 2(a) the light-emitting part 5 is connected via an optical fiber F to the power supply part 7. In FIG. 2(b) Detecting means H′ is composed of the light-emitting part 5 and the light-receiving part 9 both disposed on the same side of the optical cell 1 and a reflecting mirror M placed on the opposite side so that the light-receiving part 9 receives the ultraviolet rays G′ reflected by the mirror M.

The light-emitting part 5 is formed, for example, by an LED element that emits ultraviolet rays. FIG. 3 shows the LED emission spectrum. As will be seen from the graph, the relative emitted light intensity is 1.0 {au} when the light wavelength λ is in the range from 350 to 375 {nm}. The light-receiving part 9 is formed, for example, by a semiconductor UV sensor {a photodiode for ultraviolet rays}. Its spectral sensitivity characteristic is shown in FIG. 4. As seen from FIG. 4, the light-receiving sensitivity ranges from 20 to 60 {mA/w} when the light wavelength is 260 to 390 {nm}. FIG. 5 shows the relationship between the concentration of the antiseptic solution and the output voltage {V} when the light-receiving part 9 is used. The output voltage {V} is provided from the light-receiving part 9 that receives ultraviolet rays from the optical cell 1 filled with an antiseptic solution of a known concentration. The calibration curve K1 in FIG. 5 establishes the proportional relationship that the output voltage is 0.82 to 0.62 {V} when the concentration of the effective component of an OPA antiseptic solution is 0.1 to 0.55 {%}. FIG. 6 shows a characteristic that the concentration of the antiseptic solution is 0.55 to 0.0 {%} when the output value (recorded value) is 0.8 to 1.2 Vx/ref. The calibration curve K2 establishes the proportional relationship that the output voltage is 0.8 to 1.1 {V} when the concentration of the antiseptic solution is 0.0 to 0.55 {%}.

The antiseptic-solution concentration measuring equipment 100 of the above configuration conducts the measurement of the concentration of the antiseptic solution S such as the glutaral and phtharal solutions. A description will be given of measuring functions of the equipment. In the first place, the antiseptic solution S to be measured is poured into the optical cell 1, and the ultraviolet absorbance of the antiseptic solution S is detected by the light-receiving part 9 that receives the ultraviolet rays G emitted from the light-emitting part 5. That is, the light-receiving part 9 receives the ultraviolet rays G′ partly absorbed by the antiseptic solution S in proportion to its concentration, detects the ultraviolet absorbance of the antiseptic solution S, and outputs the electrical signal E1. The temperature of the antiseptic solution S in the optical cell 1 is measured by a thermometer and converted to the electrical signal E2. Based on the relationships between the electrical signals E1 and E2 and the prestored calibration curves, the computer CPU calculates the concentration N of the antiseptic solution S, which is displayed on the display 20.

Incidentally, prior to the above-described measurement of concentration, an used or new antiseptic solution S and pure water W are poured into the optical cell 1, then electrical signals corresponding to them, respectively, are input to the computer CPU, which uses them to perform calibration, thereby ensuring accurate measurement of the antiseptic solution S without being affected by external disturbance.

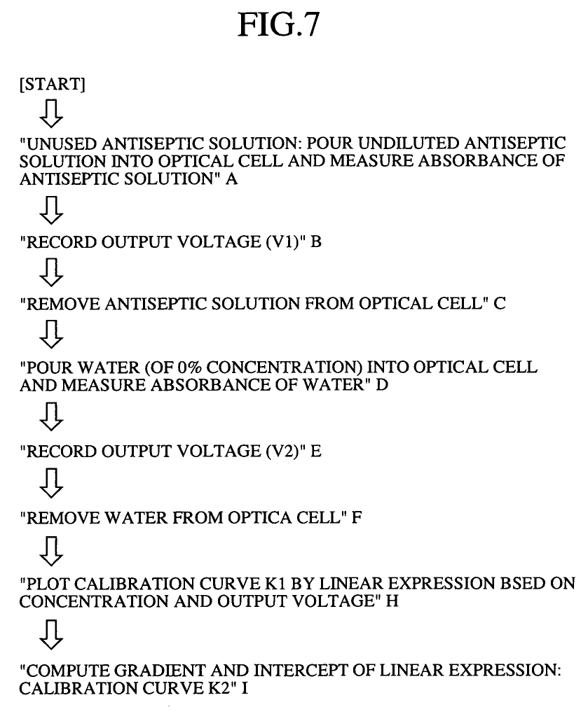

Next, a description will be given below of calibration and concentration measuring methods by the antiseptic-solution concentration measuring equipment 100. The calibration according to a first embodiment of the invention is carried out as shown in FIG. 7 which is a calibration flowchart. In the first place, prepare an antiseptic solution (the indicated concentration of a commercially available undiluted antiseptic solution is assumed as positive) and water (concentration: 0%). In step A, “pour the undiluted antiseptic solution into the optical cell and measure the absorbance of the antiseptic solution.” In step B, “record the output voltage (V1),” In step C, “remove the antiseptic solution from the optical cell.” In step D, “pour the water (of 0% concentration) into the optical cell and measure the absorbance of the water.” In step E, “record the output voltage (V2).” In step F, “remove the water from the optical cell.” In step H, “plot the calibration curve K1 by a linear expression based on the concentration and the output voltage (In FIG. 5, X axis represents voltage and Y axis concentration).” Alternatively, in step I, “compute the gradient and intercept of the linear expression (FIG. 6: calibration curve K2).”

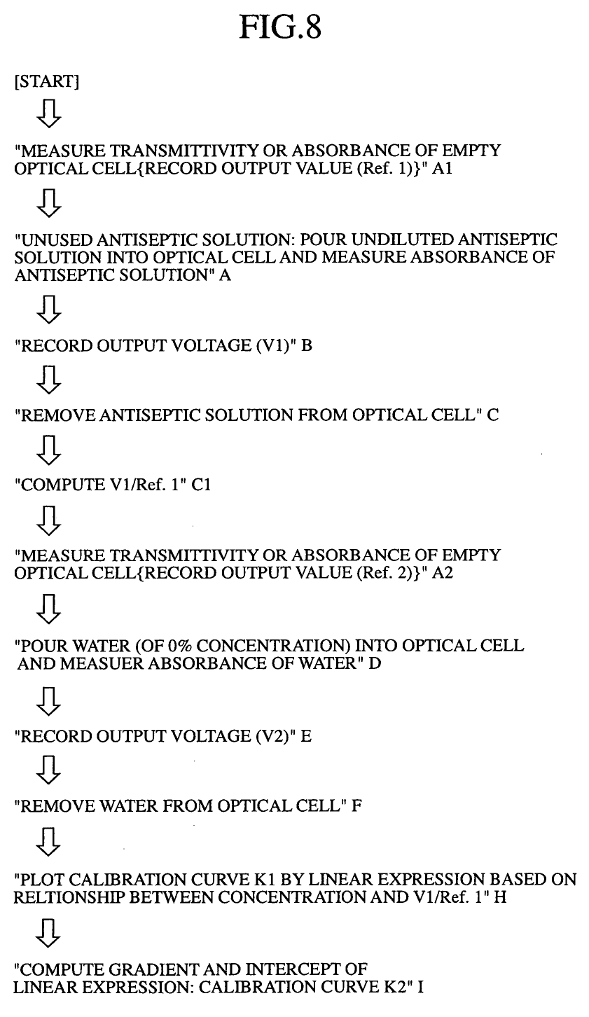

Next, the calibration method according to a second embodiment of the invention will be described with reference to FIG. 8. This calibration method utilizes an additional function for reducing measurement errors caused by electrical fluctuations in the measuring equipment (variations in the output from the amplifier due to its temperature characteristic, variations in the quantity of light from the light source, and so on) during measurement.

In the first place, prepare an antiseptic solution (the indicated concentration of a commercially available undiluted antiseptic solution is assumed as positive) and water (of 0% concentration). In step A1, “measure the transmittance of an empty optical cell {record the output value (Ref. 1)}. In step A, “pour the undiluted antiseptic solution into the optical cell and measure the absorbance of the antiseptic solution.” In step B, “record the output voltage (V1).” In step C, “remove the antiseptic solution from the optical cell.” In step C1, “compute V1/Ref. 1.” In step A2, “measure the transmittance of the empty optical cell {record the output value (Ref. 2)}. In step D, “pour water (of 0% concentration) into the optical cell and measure the absorbance of the water.” In step E, “record the output voltage (V2).” In step F, “remove the water from the optical cell.” In step H, “plot the calibration curve K2 by a linear expression based on the relationship between concentration and V1/Ref. 1 In FIG. 5, X axis represents voltage and Y axis concentration).” Alternatively, in step I “compute the gradient and intercept of the linear expression (FIG. 6, calibration curve K2).

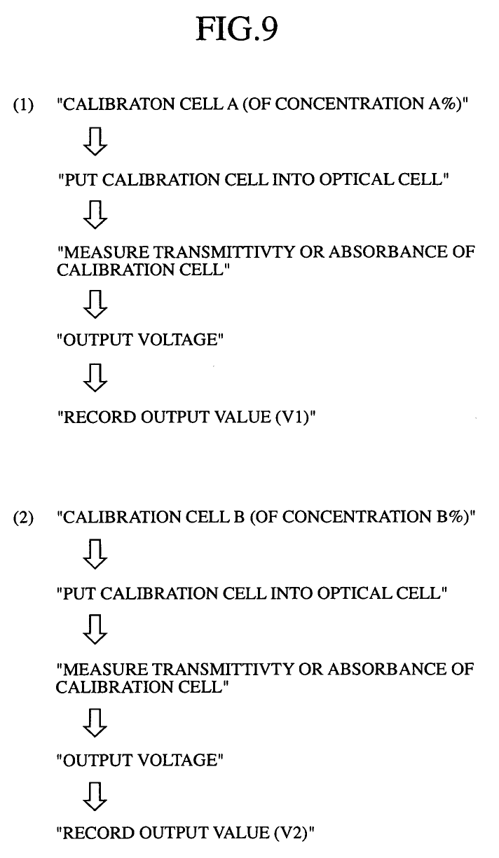

Next, the calibration method according to a third embodiment of the invention will be described with reference to FIG. 9. This calibration method uses, as a substitute for the antiseptic solution for calibration, at least two kinds of concrete objects (glass and plastics plates, and the like, which will hereinafter be referred to as calibration cells) U which has the same transmittance (or absorbance) as that of the antiseptic solution S of a known concentration. Incidentally, it is necessary that two or more kinds of concrete objects U enable the calibration curve to be plotted. One of the concrete objects may be equal in transmittance (or absorbance) to water. In the concentration measuring equipment the antiseptic solution S or water W poured into the optical cell in the first and second embodiments are substituted with the calibration cell.

[Example of Measurement]

(1) “Put calibration cell A (of A % concentration) in optical cell,” then “Measure transmittance or absorbance of calibration cell A,” then “Output voltage,” and “Record output voltage (V1).”

(2) “Put calibration cell B (of B % concentration) in optical cell,” then “Measure transmittance or absorbance of calibration cell B,” then “Output voltage,” and “Record output voltage (V2).”

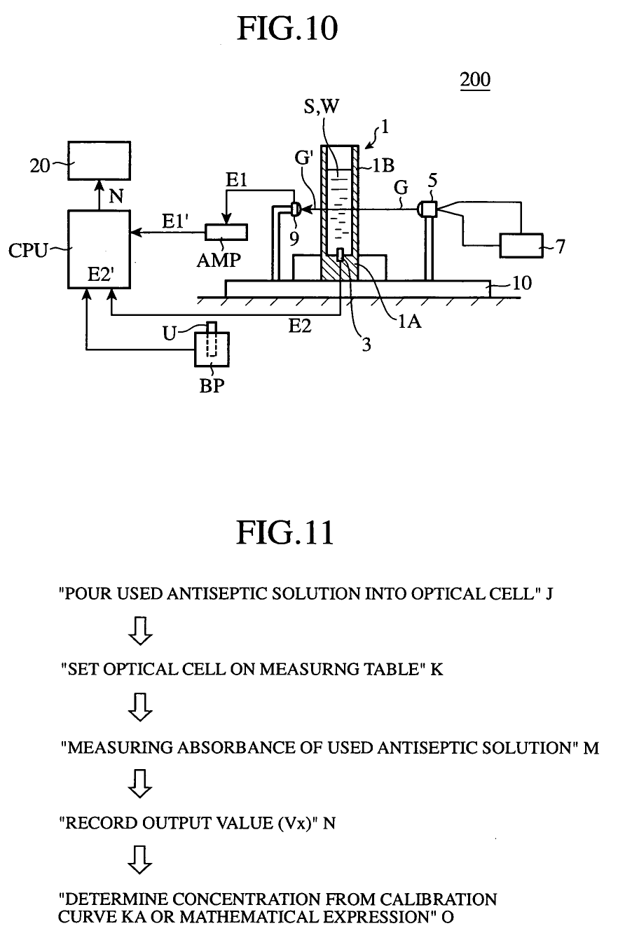

The calibration method according to a fourth embodiment of the invention will be described with reference to FIG. 10. In the first to third embodiments, an operator manually pours the antiseptic solution S or water W, or the concrete objects (the calibration cell) U into and removes them from the optical cell 1. In this embodiment, to automate such works, the concrete objects (calibration cell) U used in the embodiment 3 is not mounted on the table 10 but instead it is always located on an optical or electrical bypass circuit BP to perform automatic calibration of the measuring equipment.

The calibration of the measuring equipment is followed by the measurement of the concentration of used antiseptic solution.

(1) The simplest concentration measuring method begins with pouring the used antiseptic solution S of unknown concentration into the optical cell 1, followed by recording the output voltage E1 fed from the light-receiving part 9. The concentration of the used antiseptic solution can be inferred from the calibration curve K1 shown in FIG. 5. The procedure of this method is shown in FIG. 11: “Pour used antiseptic solution into optical cell” in step J; “Set optical cell on measuring table” in step K; “Measure absorbance of used antiseptic solution” in step L; “Output voltage” in step M; “Record output voltage Vx)” in step N; and “Measure concentration based on calibration curve K1 or mathematical expression” in step O.

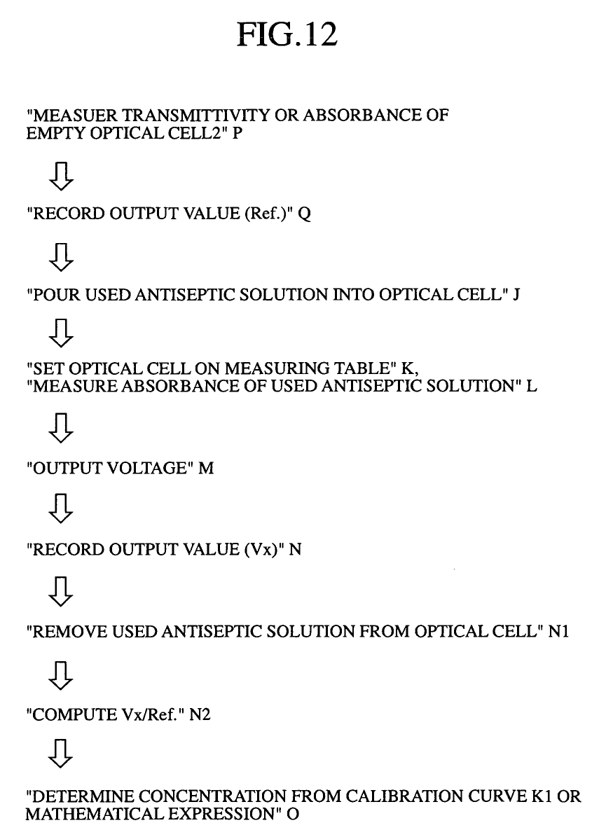

(2) FIG. 12 is a detailed flowchart showing the procedure of measurement by the above measuring equipment: “Measure transmittance or absorbance of empty optical cell” in step P; “Record output value (Ref.)” in step Q; “Pout used antiseptic solution into optical cell” in step J; “Set optical cell on measuring able” in step K; “Measure absorbance of used antiseptic solution” in step L; “Output voltage” in step M; “Record output value (Vx) in step N; “Remove antiseptic solution from optical cell” in step N1; “Compute Vx/Ref.” In step N2; and “Measure concentration based on calibration curve K1 or mathematical expression” in step O.

(3) This method automates the computation of the calibration curve K1 or K2 in FIG. 5 or 6, or the mathematical expression by the computer function of the computer CPU, and the measured concentration of the antiseptic solution S is automatically calculated from the output value and is displayed on the display 20.

(4) Furthermore, each of the above-described measuring methods measures the temperature of the antiseptic solution by the thermometer 3 mounted in the optical cell 1 to compensate for variations in the transmittance (or absorbance) by temperature, providing increased accuracy in the concentration measurement.

As described above, the mode of working of the present invention produces such effects as mentioned below. The measuring method of the present invention is very simple and easy as compared with the conventional spectrometry and the measuring equipment is also smaller than in the past. Since the amount of antiseptic solution necessary for measurement is small, exposure by the antiseptic solution can be reduced. And, the thermometer mounted in the measuring equipment enables the temperature characteristic of the absorbance to be corrected and calibrated. Furthermore, the calibration of the measuring equipment by use of unused antiseptic solution increases the measurement accuracy and enhances its reliability.

Incidentally, the present invention should not be construed as being limited specifically to the above-described embodiments. For example, individual configurations of the light-emitting part 5, the light-receiving part 9 and the optical cell 1 and their associated arrangements, and the system of the display 10 can be properly modified.

### INDUSTRIAL APPLICABILITY

The present invention has been described as being applied to the measurement of the concentration of the antiseptic solution, but the invention is not limited specifically thereto and is applicable to various other concentration measurements.

### BRIEF DESCRIPTION OF THE DRAWINGS

[FIG. 1] is a block diagram illustrating the antiseptic-solution concentration measuring equipment according to the present invention.

[FIG. 2] shows in cross-section different configurations of a light-emitting and a light-receiving part placed outside an optical cell.

[FIG. 3] is a graph showing the emission spectrum of the light-emitting part.

[FIG. 4] is a graph showing the spectral sensitivity of the light-receiving part.

[FIG. 5] is a graph showing the relationship between the concentration of the antiseptic solution under measurement and the output voltage from the light-receiving part.

[FIG. 6] is a graph showing the relationship between output value records (recorded value) and the antiseptic-solution concentration.

[FIG. 7] is a flowchart showing the procedure of a first calibration method.

[FIG. 8] is a flowchart showing the procedure of a second calibration method.

[FIG. 9] is a flowchart showing the procedure of a second calibration method.

[FIG. 10] is a block diagram illustrating concentration measuring equipment designed for a fourth calibration method.

[FIG. 11] is a flowchart showing the procedure of a first measuring method.

[FIG. 12] is a flowchart showing the procedure of a second measuring method.

### EXPLANATION OF NUMERALS

1: Optical cell
1A: Bottom
3: Thermometer
5: Light-emitting part
7: Power supply part
9: Light-receiving part
10: Measuring table
20: Display
100: Antiseptic-solution concentration measuring equipment
AMP: Amplifier
CPU: Computer
E1, E1′: Electrical signal representing absorbance
E2, E2′: Electrical signal representing temperature
F: Optical fiber
G, G′: Ultraviolet rays
H, H′: Detecting means
N: Concentration value
M: Reflecting mirror
S: Antiseptic Solution
W: Pure water

1. A method for measuring the concentration of an antiseptic solution, comprising the steps of electrically measuring the absorbance of said antiseptic solution, such as a glutaral or phtharal solution, by a photodetector to detect its absorbance of ultraviolet rays; and calculating, by a computer, a function of proportionality between the measured ultraviolet absorbance of said antiseptic solution and pre-measured ultraviolet absorbance of an undiluted antiseptic solution and a function of proportionality between the ultraviolet absorbance and concentration of said undiluted antiseptic solution.

2. A method for measuring the concentration of an antiseptic solution, comprising measuring the temperature of said antiseptic solution, such as a glutaral or phtharal solution, by a thermometer; electrically measuring the absorbance of said antiseptic solution by a photodetector to detect its absorbance of ultraviolet rays; and calculating, by a computer, a function of proportionality between the ultraviolet absorbance of said antiseptic solution and pre-measured ultraviolet absorbance of an undiluted antiseptic solution and a function of proportionality between the ultraviolet absorbance and concentration of said undiluted antiseptic solution.

3. The method of claim 1, further including the steps of: pouring an unused antiseptic solution and pure water into the optical cell separately of each other; calibrating, by a computer, electrical signals from the optical cell which represent the ultraviolet absorbance and concentration of said unused antiseptic solution and the pure water, respectively; and calibrating said concentration measuring equipment based on the calibrated values.

4. Equipment for measuring the concentration of an antiseptic solution, comprising an optical cell for containing an antiseptic solution under measurement; a light-emitting part for irradiating the antiseptic solution in said optical cell with ultraviolet rays; a light-receiving part provided outside said optical cell, for receiving ultraviolet rays partly absorbed by said antiseptic solution in proportion to the concentration thereof, for detecting the ultraviolet absorbance of said antiseptic solution from the intensity of the received ultraviolet rays, and for generating an electrical signal representing the ultraviolet absorbance of said antiseptic solution; a computer for calculating the concentration value of said antiseptic solution by calculating the relationships between said electrical signal and pre-input electrical signals representing the ultraviolet absorbance and concentration of an undiluted antiseptic solution; and a display for displaying said concentration value calculated by said computer.

5. Equipment for measuring the concentration of an antiseptic solution, comprising an optical cell for containing an antiseptic solution under measurement; a light-emitting part for irradiating the antiseptic solution in said optical cell with ultraviolet rays; a light-receiving part provided outside said optical cell, for receiving ultraviolet rays partly absorbed by said antiseptic solution in proportion to the concentration thereof, for detecting the ultraviolet absorbance of said antiseptic solution from the intensity of the received ultraviolet rays, and for generating an electrical signal representing the ultraviolet absorbance of said antiseptic solution; a thermometer for measuring the temperature of the antiseptic solution in said optical cell, and for converting said measured temperature to the corresponding electrical signal; a computer for calculating the concentration value of said antiseptic solution by calculating the relationships between said electrical signals representing the ultraviolet absorbance and temperature of said antiseptic solution and pre-input electrical signals representing the absorbance and concentration of an undiluted antiseptic solution; and a display for displaying said concentration value calculated by said computer.

6. The equipment of claim 4, wherein an unused antiseptic solution and pure water are separately poured into the optical cell; and electrical signals from said optical cell which represent the ultraviolet absorbance and concentration of said unused antiseptic solution and the pure water, respectively, are calibrated by said computer; and said measuring equipment is calibrated based on the calibrated value.

7. The method of claim 2, further including the steps of: pouring an unused antiseptic solution and pure water into the optical cell separately of each other; calibrating, by a computer, electrical signals from the optical cell which represent the ultraviolet absorbance and concentration of said unused antiseptic solution and the pure water, respectively; and calibrating said concentration measuring equipment based on the calibrated values.

8. The equipment of claim 5, wherein an unused antiseptic solution and pure water are separately poured into the optical cell; and electrical signals from said optical cell which represent the ultraviolet absorbance and concentration of said unused antiseptic solution and the pure water, respectively, are calibrated by said computer; and said measuring equipment is calibrated based on the calibrated value.
